(19) United States
(12) Patent Application Publication
(10) Pub. No.: US 2024/0423580 A1
(43) Pub. Date: Dec. 26, 2024

(54) AORTIC STENOSIS SEVERITY QUANTIFICATION USING HEART SOUNDS

(71) Applicant: Cardiac Pacemakers, Inc., St. Paul, MN (US)

(72) Inventors: Mojgan Goftari, Shoreview, MN (US); Viktoria A. Averina, Shoreview, MN (US); Pramodsingh Hirasingh Thakur, Woodbury, MN (US); Jonathan Bennett Shute, Eagan, MN (US)

(21) Appl. No.: 18/752,446

(22) Filed: Jun. 24, 2024

Related U.S. Application Data
(60) Provisional application No. 63/523,152, filed on Jun. 26, 2023.

(51) Int. Cl. A61B 7/04; A61B 5/00; A61B 5/0205; A61B 5/024; A61B 5/08; A61B 5/11; A61B 5/28; G16H 20/40; G16H 50/20; G16H 50/30

(52) U.S. Cl. CPC A61B 7/04; A61B 5/0205; A61B 5/72; G16H 20/40; G16H 50/20; G16H 50/30; A61B 5/024; A61B 5/08; A61B 5/11; A61B 5/28

(57) ABSTRACT

Systems and methods for monitoring and managing aortic stenosis are disclosed. An exemplary medical-device system comprises a data receiver to receive heart sound information sensed by ambulatory heart sound sensors, and a stenosis detector to detect a change in aortic valve surface area from a baseline stenosis-free state using heart sound components such as S1 and S2 sounds. Based on the change in aortic valve surface area, the stenosis detector can generate an aortic stenosis indicator indicating a presence and severity of aortic stenosis. Such indicator can be provided to a user, trigger symptom monitoring and evaluation of functional deterioration in the patient, facilitate assessment of patient candidacy for aortic valve replacement, or triage heart failure management strategies.

### CLAIM OF PRIORITY

This application claims the benefit of U.S. Provisional Application No. 63/523,152 filed on Jun. 26, 2023, which is hereby incorporated by reference in its entirety.

### TECHNICAL FIELD

This document relates generally to medical systems, and more particularly, to systems, devices and methods for detecting aortic stenosis in a patient using heart sound information.

### BACKGROUND

Aortic stenosis, also known as aortic valve stenosis, is a major form of cardiovascular diseases that accounts for over 60% of valvular heart disease (VHD). Aortic stenosis typically occurs when the aortic valve narrows and blood cannot flow normally. Over time, aortic stenosis may cause the left ventricle of the heart to pump harder to push blood through the narrowed aortic valve, which may further cause the left ventricle to thicken, enlarge, and weaken. If not timely and properly addressed and corrected, aortic stenosis may lead to heart failure.

Heart sounds are generally associated with mechanical vibration of a heart and blood flow through the heart. Heart sounds recur with each cardiac cycle and are separated and classified according to the activity associated with the vibration. Historically, heart sounds were assessed by humans and thus only audible portion of the vibration was used. Devices can now assess full spectrum of cardiac vibrations which include both audible and subaudible components, thus the term “sound” generally refers to the full spectrum of vibrations. Typically, heart sounds sensed from a subject may include several components within a cardiac cycle, including a first (S1), a second (S2), a third (S3), or a fourth (S4) heart sound. S1 is associated with the vibrations produced by the heart during tensing of the mitral valve. S2 is produced by closure of the aortic and pulmonary valves, and marks the beginning of diastole. S3 is produced by early diastolic vibrations corresponding to passive ventricular filling during diastole, when the blood rushes into the ventricles. S4 is produced by late diastolic vibrations corresponding to active ventricular filling when the atria contract and push the blood into the ventricles. In a healthy subject, S3 is usually faint and S4 is rarely audible. However, a pathologic S3 or S4 may be higher pitched and louder.

Heart sounds have been used to assess cardiac systolic and diastolic functions. Systole is the contraction or a period of contraction of the heart that causes blood to be forced out of the heart such as the ventricles and into the aorta and pulmonary artery. Diastole is the relaxation or a period of relaxation of the heart during which the blood flows back into the heart such as the ventricles. Patients with heart failure may have deteriorated systolic or diastolic functions.

### Overview

Aortic stenosis can be graded as mild, moderate, severe or critical. The rate of progression may vary depending on patient overall health, symptoms, aortic valve leaflet anatomy (e.g., thickening and calcification of the leaflets) and mobility, cardiac (particularly left-ventricular) function, the stage at which the patient is diagnosed the treatment received, among other factors. Although many patients with severe aortic stenosis are presented with various symptoms including fatigue, shortness of breath, chest pain, or irregular heartbeats (e.g., rapid fluttering heartbeats), a substantial number of severe aortic stenosis patients are asymptomatic at the time of first diagnosis. However, although aortic stenosis may remain asymptomatic for decades, once symptoms occur, the survival can be severely compromised. Early detection and classification of aortic stenosis and proper treatment, including balloon valvuloplasty or valve replacement procedure such as transcatheter aortic valve replacement (TAVR) or surgical aortic valve replacement (SAVR), can reduce mortality and improve outcome.

Conventional aortic stenosis diagnosis generally requires periodic transthoracic echocardiogram to understand anatomical and hemodynamic changes of aortic valve. Other cardiovascular imaging studies such as computerized tomography (CT) scan, magnetic resonance imaging (MRI), and transesophageal echocardiogram (TEE), and cardiac catheterization may also be used in diagnosing and evaluating the severity of aortic stenosis. Based on the imaging studies, symptoms, and cardiac functions, aortic stenosis may be categorized into one of four stages: a first stage of being at risk of stenosis, a second stage of progressive (or mild to moderate) stenosis, a third stage of asymptomatic severe stenosis, and a fourth stage of symptomatic severe stenosis. Early stages of aortic stenosis are typically characterized by changes in aortic valve anatomy, including reduced surface area of aortic valve and leaflet thickening and calcification. Although echocardiogram has been regarded as a standard test for diagnosing aortic stenosis, it generally provides a “snapshot” of the valve anatomy and function at the time of testing but does not track aortic stenosis progression over time. Periodic or frequent echocardiogram test can help track aortic stenosis deterioration, but it is costly and for some patients can be inconvenient. This may compromise patient compliance, and delay the treatment until aortic stenosis deteriorates and becomes symptomatic. The present inventors have recognized an unmet need for medical systems, devices, and techniques for ambulatory monitoring of aortic stenosis patients, diagnosing aortic stenosis using effective yet less expensive sensors to improve early detection and quantification of severity of aortic stenosis, thereby preventing or slowing deterioration to severe symptomatic aortic stenosis in such patients.

The present document discusses systems, devices, and methods for monitoring a valvular heart disease such as aortic stenosis. An exemplary medical-device system comprises a data receiver to receive heart sound information sensed by ambulatory heart sound sensors, and a stenosis detector to detect a change in aortic valve surface area from a baseline stenosis-free state using heart sound components such as S1 and S2 sounds. Based on the change in aortic valve surface area, the stenosis detector can generate an aortic stenosis indicator indicating a presence and severity of aortic stenosis. Such indicator can be provided to a user, trigger symptom monitoring and evaluation of functional deterioration in the patient, facilitate assessment of patient candidacy for aortic valve replacement, or triage heart failure management strategies.

Example 1 is a medical-device system for managing a valvular heart disease, comprising: a data receiver circuit configured to receive heart sound information sensed from a patient; and a controller circuit, comprising a stenosis detector circuit configured to: detect heart sound components including S1 sound and S2 sound from the received heart sound information; determine a change in aortic valve surface area from a baseline stenosis-free state using the detected S1 and S2 sounds; and generate an aortic stenosis indicator based at least in part on the determined change in aortic valve surface area, wherein the controller circuit is configured to provide the aortic stenosis indicator to a user or a process executable by the medical-device system.

In Example 2, the subject matter of Example 1 optionally includes, wherein to determine the change in aortic valve surface area includes to detect a decrease in aortic valve surface area from the baseline stenosis-free state, wherein the stenosis detector is configured to generate the aortic stenosis indicator indicating a presence of aortic stenosis in response to the decrease in aortic value surface area exceeding a threshold.

In Example 3, the subject matter of any one or more of Examples 1-2 optionally include, wherein to determine the change in aortic valve surface area, the stenosis detector is configured to: generate an estimate of a left ventricular ejection time using a time interval between the detected S1 and S2 sounds, and an estimate of an aorta-to-left ventricular pressure gradient using an intensity of the S2 sound; and determine the aortic valve surface area based on the estimate of the aorta-to-left ventricular pressure gradient and the estimate of the left ventricular ejection time.

In Example 4, the subject matter of Example 3 optionally includes the stenosis detector that can be configured to determine the aortic valve surface area to be inversely proportional to the estimate of the left ventricular ejection time.

In Example 5, the subject matter of any one or more of Examples 3-4 optionally includes the stenosis detector that can be configured to determine the aortic valve surface area to be inversely proportional to a square root of the estimate of the aorta-to-left ventricular pressure gradient.

In Example 6, the subject matter of any one or more of Examples 1-5 optionally includes the controller circuit that can be configured to generate an alert to the user in response to the aortic stenosis indicator indicating a presence of aortic stenosis.

In Example 7, the subject matter of any one or more of Examples 1-6 optionally includes the controller circuit that can be configured to, in response to the aortic stenosis indicator indicating a presence of aortic stenosis: trigger the data receiver circuit to receive symptom or physiological information of the patient indicative of functional deterioration associated with the presence of aortic stenosis; and categorize a stenosis severity level based on the aortic stenosis indicator and the received symptom or physiological information.

In Example 8, the subject matter of Example 7 optionally includes a user interface coupled to data receiver circuit, the user interface configured to receive a user input of symptom in response to the indication of the presence of aortic stenosis.

In Example 9, the subject matter of any one or more of Examples 7-8 optionally includes the triggered received physiological information that can include an intensity of S1 sound indicative of cardiac contractility.

In Example 10, the subject matter of any one or more of Examples 7-9 optionally includes one or more sensors coupled to data receiver circuit and configured to sense physiological information in response to the indication of the presence of aortic stenosis, the one or more sensors including at least one of a physical activity sensor, a respiration sensor, a heart rate sensor, or an electrocardiogram sensor.

In Example 11, the subject matter of any one or more of Examples 1-10 optionally includes the controller circuit that can be configured to generate an indicator of patient candidacy for aortic valve replacement procedure based at least in part on the aortic stenosis indicator.

In Example 12, the subject matter of Example 11 optionally includes the controller circuit that can be configured receive symptom or physiological information of the patient indicative of functional deterioration associated with the aortic stenosis, and to generate the indicator of patient candidacy for aortic valve replacement further based on the received symptom or physiological information.

In Example 13, the subject matter of any one or more of Examples 1-12 optionally includes the controller circuit that includes a heart failure detector circuit configured to detect worsening heart failure (WHF) using physiological information received from the data receiver circuit.

In Example 14, the subject matter of Example 13 optionally includes the controller circuit that can be configured to, in response to the aortic stenosis indicator indicating a presence of aortic stenosis, generate a diagnosis of WHF secondary to aortic stenosis, and provide the diagnosis to the user.

In Example 15, the subject matter of Example 14 optionally includes the controller circuit that can be configured to generate a recommendation to titrate therapy based on the diagnosis of WHF secondary to aortic stenosis.

Example 16 is a method of managing a valvular heart disease using a medical-device system, the method comprising: receiving heart sound information sensed from a patient; and detecting heart sound components including S1 sound and S2 sound from the received heart sound information; determining, via a stenosis detector circuit, a change in aortic valve surface area from a baseline stenosis-free state using the detected S1 and S2 sounds; generating an aortic stenosis indicator based at least in part on the determined change in aortic valve surface area; and providing the aortic stenosis indicator to a user or a process executable by the medical-device system.

In Example 17, the subject matter of Example 16 optionally includes determining the change in aortic valve surface area includes detecting a decrease in aortic valve surface area from the baseline stenosis-free state, wherein the aortic stenosis indicator indicates a presence of aortic stenosis when the decrease in aortic value surface area exceeds a threshold.

In Example 18, the subject matter of any one or more of Examples 16-17 optionally includes determining the change in aortic valve surface area that can include: generating an estimate of a left ventricular ejection time using a time interval between the detected S1 and S2 sounds, and generating an estimate of an aorta-to-left ventricular pressure gradient using an intensity of the S2 sound; and determining an aortic valve surface area based on the estimate of the aorta-to-left ventricular pressure gradient and the estimate of the left ventricular ejection time.

In Example 19, the subject matter of any one or more of Examples 16-18 optionally include generating an alert to the user in response to the aortic stenosis indicator indicating a presence of aortic stenosis.

In Example 20, the subject matter of any one or more of Examples 16-19 optionally includes, in response to the aortic stenosis indicator indicating a presence of aortic stenosis: receiving symptom or physiological information of the patient indicative of functional deterioration associated with the presence of aortic stenosis; and categorizing a stenosis severity level based on the aortic stenosis indicator and the received symptom or physiological information.

In Example 21, the subject matter of Example 20 optionally includes receiving the symptom or physiological information of the patient that can include receiving a user input of symptom, or sensing physiological information using one or more sensors including at least one of a heart sound sensor, physical activity sensor, a respiration sensor, a heart rate sensor, or an electrocardiogram sensor.

In Example 22, the subject matter of any one or more of Examples 16-21 optionally includes generating, and providing to a user, an indicator of patient candidacy for aortic valve replacement procedure based at least in part on the aortic stenosis indicator.

In Example 23, the subject matter of any one or more of Examples 16-22 optionally includes: detecting worsening heart failure (WHF) using physiological information; and in response to the aortic stenosis indicator indicating a presence of aortic stenosis, generating a diagnosis of WHF secondary to aortic stenosis, and generating a recommendation to titrate therapy based on the diagnosis of WHF secondary to aortic stenosis.

The heart sound (HS)-based aortic stenosis detection as described in this disclosure may improve the functionality of an ambulatory medical device, or a medical diagnostic system or device, which uses heart sounds to monitor and managing patients with aortic stenosis. In contrast to conventional echocardiogram-based stenosis diagnosis, the HS-based aortic stenosis detection as described herein allows early detection and evaluation of aortic stenosis in an ambulatory setting, reduces time and burden associated with frequent echocardiogram check to track stenosis progression in a clinical setting, and saves cost. In various embodiments, the HS-based aortic stenosis detection involves determining a change in aortic valve surface area using heart sound components. The aortic valve surface area is conventionally measured during a cardiac catheterization procedure, which poses risks to patient in addition to clinic visit, possible hospitalization, and additional cost. The present document describes non-invasive, non-catheter, HS-based approaches to effectively and efficiently estimate a change in aortic valve surface area in an ambulatory setting. The heart sounds can be collected by an ambulatory sensor, such as one embedded in an implantable medical device. With the use of heart sounds in aortic stenosis diagnosis, lower cost or less obtrusive systems, apparatus, and methods may be used. It may particularly be beneficial for patients at early stage of aortic stenosis or those constrained by accessibility, scheduling, affordability, or various contraindications to cardiac catheterization procedure. The systems, devices, and techniques as described herein also improves identifying patient candidacy for valve replacement procedure (e.g., TAVR or SAVR), and preventing or slowing the deterioration to severe symptomatic aortic stenosis.

The efficient HS-based stenosis detection as described in this disclosure can also improve power and resource usage in a medical device of system. A technological problem exists in medical devices and medical device systems that in low-power monitoring modes, ambulatory medical devices powered by one or more rechargeable or non-rechargeable batteries (e.g., including IMDs) have to make certain tradeoffs between battery life, or in the instance of implantable medical devices with non-rechargeable batteries, between device replacement periods often including surgical procedures, and sampling resolution, sampling periods, of processing, storage, and transmission of sensed physiologic information, or features or mode selection of or within the medical devices. Medical devices can include higher-power modes and lower-power modes. Physiologic information, such as indicative of a potential adverse physiologic event, can be used to transition from a low-power mode to a high-power mode. In certain examples, the low-power mode can include a low resource mode, characterized as requiring less power, processing time, memory, or communication time or bandwidth (e.g., transferring less data, etc.) than a corresponding high-power mode. The high-power mode can include a relatively higher resource mode, characterized as requiring more power, processing time, memory, or communication time or bandwidth than the corresponding low-power mode. However, by the time physiologic information detected in the low-power mode indicates a possible event, valuable information has been lost, unable to be recorded in the high-power mode. The inverse is also true, in that false or inaccurate determinations that trigger a high-power mode unnecessarily unduly limit the usable life of certain ambulatory medical devices. The HS-based aortic stenosis detection as described herein allows for more accurate detection and determination of stenotic valve and physiologic events (e.g., WHF) secondary to stenosis, thereby avoiding unnecessary transitions from the low-power mode to the high-power mode and improving use of medical device resources and power.

This Overview provides some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates generally an example of a patient management system and portions of an environment in which the system may operate.

FIG. 2 illustrates generally an example of an aortic stenosis detection system to detect presence and severity of aortic stenosis using heart sounds information.

FIG. 3 illustrates normal aortic valve and stenotic aortic valve in open and closed states.

FIGS. 4A and 4B illustrate generally an example of changes in heart sounds in an aortic stenosis patient before and after a TAVR procedure.

FIG. 5 is a flowchart illustrating an example method of managing a valvular heart disease such as aortic stenosis.

FIG. 6 illustrates generally a block diagram of an example machine upon which any one or more of the techniques (e.g., methodologies) discussed herein may perform.

### DETAILED DESCRIPTION

Disclosed herein are systems, devices, and methods for monitoring and managing patients with valvular heart disease such as aortic stenosis. An exemplary medical-device system comprises a data receiver circuit to receive heart sound information, and a stenosis detector circuit to detect and quantify aortic stenosis using heart sounds sensed from a patient. The stenosis detector circuit can determine a change in aortic valve surface area from a baseline stenosis-free state using heart sound components such as S1 and S2 sounds, and generate an aortic stenosis indicator based on the determined change in aortic valve surface area. A controller can provide the aortic stenosis indicator to a user or a process executable by the medical-device system, such as to trigger symptom monitoring, classify aortic stenosis into different stages, facilitate assessment of patient candidacy for valve replacement procedure, or triage heart failure management strategies.

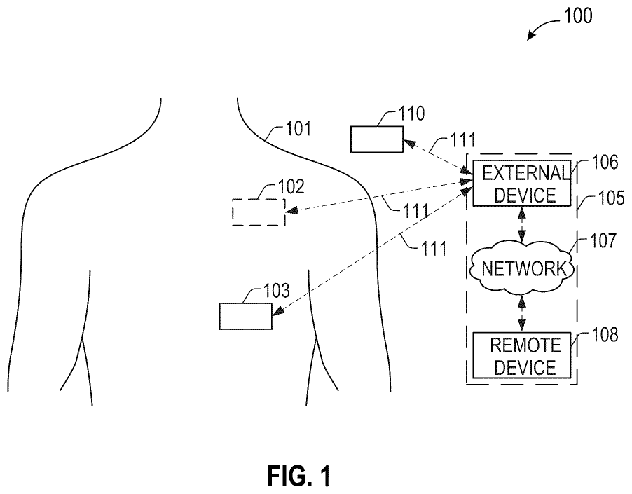

FIG. 1 illustrates generally an example patient management system 100 and portions of an environment in which the patient management system 100 may operate. The patient management system 100 can perform a range of activities, including remote patient monitoring and diagnosis of a disease condition, such as aortic stenosis or other valvular heart diseases. Such activities can be performed proximal to a patient 101, such as in a patient home or office, through a centralized server, such as in a hospital, clinic, or physician office, or through a remote workstation, such as a secure wireless mobile computing device.

The patient management system 100 may include one or more ambulatory medical devices, an external system 105, and a communication link 111 providing for communication between the one or more ambulatory medical devices and the external system 105. The one or more ambulatory medical devices may include an implantable medical device (IMD) 102, a wearable medical device (WMD) 103, or one or more other implantable, leadless, subcutaneous, external, wearable, or ambulatory medical devices configured to monitor, sense, or detect information from, determine physiologic information about, or provide one or more therapies to treat various conditions of the patient 101, such as one or more cardiac or non-cardiac conditions (e.g., dehydration, sleep disordered breathing, etc.).

In an example, the IMD 102 may include one or more traditional cardiac rhythm management devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 101. In another example, the IMD 102 may include a monitor implanted, for example, subcutaneously in the chest of patient 101, the IMD 102 including a housing containing circuitry and, in certain examples, one or more sensors, such as a temperature sensor, etc.

The IMD 102 may include an assessment circuit configured to detect or determine specific physiologic information of the patient 101, or to determine one or more conditions or provide information or an alert to a user, such as the patient 101 (e.g., a patient), a clinician, or one or more other caregivers or processes. In an example, the IMD 102 can be an implantable cardiac monitor (ICM) configured to collected cardiac information, optionally along with other physiological information, from the patient. The IMD 102 can alternatively or additionally be configured as a therapeutic device configured to treat one or more medical conditions of the patient 101. The therapy can be delivered to the patient 101 via the lead system and associated electrodes or using one or more other delivery mechanisms. The therapy may include delivery of one or more drugs to the patient 101, such as using the IMD 102 or one or more of the other ambulatory medical devices, etc. In some examples, therapy may include cardiac resynchronization therapy for rectifying dyssynchrony and improving cardiac function in heart failure patients. In other examples, the IMD 102 may include a drug delivery system, such as a drug infusion pump to deliver drugs to the patient for managing arrhythmias or complications from arrhythmias, hypertension, or one or more other physiologic conditions. In other examples, the IMD 102 may include one or more electrodes configured to stimulate the nervous system of the patient or to provide stimulation to the muscles of the patient airway, etc.

The WMD 103 may include one or more wearable or external medical sensors or devices (e.g., automatic external defibrillators (AEDs), Holter monitors, patch-based devices, smart watches, smart accessories, wrist- or finger-worn medical devices, such as a finger-based photoplethysmography sensor, etc.).

In an example, the IMD 102 or the WMD 103 may include or be coupled to an implantable or wearable sensor to sense a heart sound signal, and include a heart sound recognition circuit to recognize one or more heart sound components such as S1, S2, S3, or S4 based on a spectral entropy time series derived from the sensed heart sound signal. Also included in the IMD 102 or the WMD 103 is a heart sound (HS)-based event detector circuit that can detect a physiologic event (e.g., a cardiac arrhythmia episode, or a worsening heart failure (WHF) event) based at least on a heart sound metric of the detected one or more heart sound component. Examples of such heart sound metric may include an amplitude, or timing of the heart sound component within a cardiac cycle relative to a fiducial point. In some examples, at least a portion of the heart sound recognition circuit and/or the HS-based event detector circuit may be implemented in and executed by the external system 105.

The external system 105 may include a dedicated hardware/software system, such as a programmer, a remote server-based patient management system, or alternatively a system defined predominantly by software running on a standard personal computer. The external system 105 can manage the patient 101 through the IMD 102 or one or more other ambulatory medical devices connected to the external system 105 via a communication link 111. In other examples, the IMD 102 can be connected to the WMD 103, or the WMD 103 can be connected to the external system 105, via the communication link 111. This may include, for example, programming the IMD 102 to perform one or more of acquiring physiological data, performing at least one self-diagnostic test (such as for a device operational status), analyzing the physiological data, or optionally delivering or adjusting a therapy for the patient 101. Additionally, the external system 105 can send information to, or receive information from, the IMD 102 or the WMD 103 via the communication link 111. Examples of the information may include real-time or stored physiological data from the patient 101, diagnostic data, such as detection of patient hydration status, hospitalizations, responses to therapies delivered to the patient 101, or device operational status of the IMD 102 or the WMD 103 (e.g., battery status, lead impedance, etc.). The communication link 111 can be an inductive telemetry link, a capacitive telemetry link, or a radiofrequency (RF) telemetry link, or wireless telemetry based on, for example, “strong” Bluetooth or IEEE 802.11 wireless fidelity “Wi-Fi” interfacing standards. Other configurations and combinations of patient data source interfacing are possible.

The external system 105 may include an external device 106 in proximity of the one or more ambulatory medical devices, and a remote device 108 in a location relatively distant from the one or more ambulatory medical devices, in communication with the external device 106 via a communication network 107. Examples of the external device 106 may include a medical device programmer. The remote device 108 can be configured to evaluate collected patient or patient information and provide alert notifications, among other possible functions. In an example, the remote device 108 may include a centralized server acting as a central hub for collected data storage and analysis. The server can be configured as a uni-, multi-, or distributed computing and processing system. The remote device 108 can receive data from multiple patients. The data can be collected by the one or more ambulatory medical devices, among other data acquisition sensors or devices associated with the patient 101. The server may include a memory device to store the data in a patient database. The server may include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications may include a Web page update, phone or pager call, E-mail, SMS, text or “Instant” message, as well as a message to the patient and a simultaneous direct notification to emergency services and to the clinician. Other alert notifications are possible. The server may include an alert prioritizer circuit configured to prioritize the alert notifications. For example, an alert of a detected physiologic event can be prioritized using a similarity metric between the physiological data associated with the detected physiologic event to physiological data associated with the historical alerts.

The remote device 108 may additionally include one or more locally configured clients or remote clients securely connected over the communication network 107 to the server. Examples of the clients may include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 108, including the server and the interconnected clients, may also execute a follow-up scheme by sending follow-up requests to the one or more ambulatory medical devices, or by sending a message or other communication to the patient 101 (e.g., the patient), clinician or authorized third party as a compliance notification.

The communication network 107 can provide wired or wireless interconnectivity. In an example, the communication network 107 can be based on the Transmission Control Protocol/Internet Protocol (TCP/IP) network communication specification, although other types or combinations of networking implementations are possible. Similarly, other network topologies and arrangements are possible.

One or more of the external device 106 or the remote device 108 can output the detected physiologic events to a system user, such as the patient or a clinician, or to a process including, for example, an instance of a computer program executable in a microprocessor. In an example, the process may include an automated generation of recommendations for anti-arrhythmic therapy, or a recommendation for further diagnostic test or treatment. In an example, the external device 106 or the remote device 108 may include a respective display unit for displaying the physiologic or functional signals, or alerts, alarms, emergency calls, or other forms of warnings to signal the detection of arrhythmias. In some examples, the external system 105 may include an external data processor configured to analyze the physiologic or functional signals received by the one or more ambulatory medical devices, and to confirm or reject the detection of arrhythmias. Computationally intensive algorithms, such as machine-learning algorithms, can be implemented in the external data processor to process the data retrospectively to detect cardiac arrhythmias.

Portions of the one or more ambulatory medical devices or the external system 105 can be implemented using hardware, software, firmware, or combinations thereof. Portions of the one or more ambulatory medical devices or the external system 105 can be implemented using an application-specific circuit that can be constructed or configured to perform one or more functions or can be implemented using a general-purpose circuit that can be programmed or otherwise configured to perform one or more functions. Such a general-purpose circuit may include a microprocessor or a portion thereof, a microcontroller or a portion thereof, or a programmable logic circuit, a memory circuit, a network interface, and various components for interconnecting these components. For example, a “comparator” may include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” may include electronic circuits configured to receive information and provide an electronic output representative of such received information.

The therapy device 110 can be configured to send information to or receive information from one or more of the ambulatory medical devices or the external system 105 using the communication link 111. In an example, the one or more ambulatory medical devices, the external device 106, or the remote device 108 can be configured to control one or more parameters of the therapy device 110. The external system 105 can allow for programming the one or more ambulatory medical devices and can receives information about one or more signals acquired by the one or more ambulatory medical devices, such as can be received via a communication link 111. The external system 105 may include a local external implantable medical device programmer. The external system 105 may include a remote patient management system that can monitor patient status or adjust one or more therapies such as from a remote location.

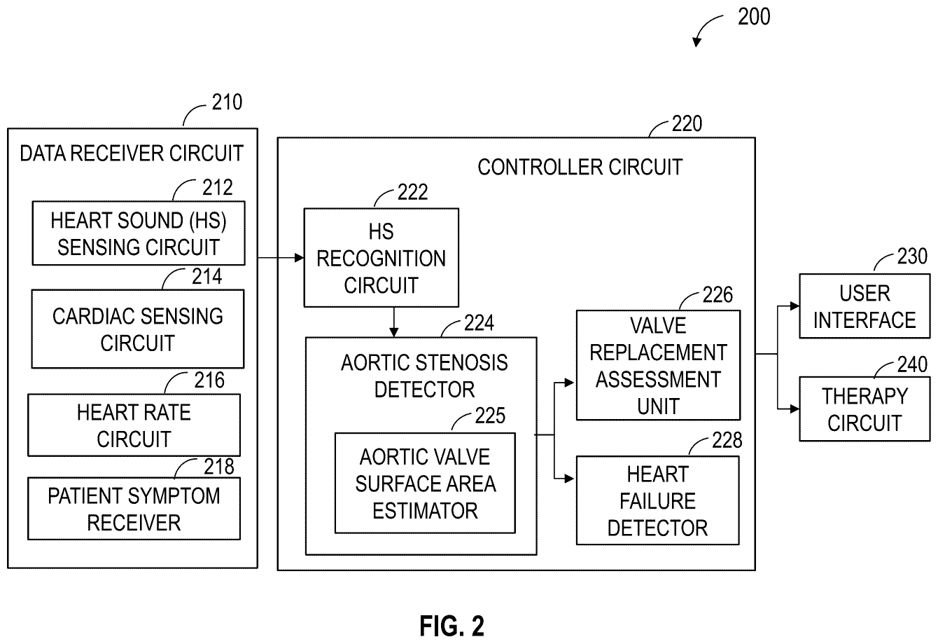

FIG. 2 illustrates generally an example of an aortic stenosis detection system 200 configured to detect presence and severity of aortic stenosis in a patient using heart sounds information. The aortic stenosis detection system 200 may include one or more of a data receiver circuit 210, a controller circuit 220, a user interface 230, and a therapy circuit 240. At least a portion of the aortic stenosis detection system 200 may be implemented in the IMD 102, the WMD 103, or the external system 105 such as one or more of the external device 106 or the remote device 108.

The data receiver circuit 210 may receive physiologic information from a patient. In an example, the data receiver circuit 210 may include a sense amplifier circuit configured to sense a physiologic signal from a patient via a physiologic sensor, such as an implantable, wearable, or otherwise ambulatory sensor or electrodes associated with the patient. The sensor may be incorporated into, or otherwise associated with an ambulatory device such as the IMD 102 or the WMD 103. In some examples, the physiologic signals sensed from a patient may be stored in a storage device, such as an electronic medical record (EMR) system. The data receiver circuit 210 may receive the physiologic signal from the storage device, such as in response to a user command or a triggering event. By way of example and not limitation and as illustrated in FIG. 2, the data receiver circuit 210 may include one or more of a heart sound sensing circuit 212, a cardiac sensing circuit 214, a heart rate circuit 216, and a patient symptom receiver 218. The heart sound sensing circuit 212 may receive heart sound information, such as a heart sound signal sensed from the patient. In an example, the heart sound sensing circuit 212 may be coupled to a heart sound sensor to sense a body motion/vibration signal indicative of cardiac vibration, which is correlated to or indicative of heart sounds. The heart sound sensor may take the form of an accelerometer, an acoustic sensor, a microphone, a piezo-based sensor, or other vibrational or acoustic sensors. The accelerometer can be a one-axis, a two-axis, or a three-axis accelerometer. Examples of the accelerometer may include flexible piezoelectric crystal (e.g., quartz) accelerometer or capacitive accelerometer, fabricated using micro electro-mechanical systems (MEMS) technology. The heart sound sensor may be included in the IMD 102 or the WMD 103, or disposed on a lead such as a part of the lead system associated with the IMD 102 or the WMD 103. In an example, an accelerometer (or other sensor types) may sense an epicardial or endocardial acceleration (EA) signal from a portion of a heart, such as on an endocardial or epicardial surface of one of a left ventricle, a right ventricle, a left atrium, or a right atrium. The EA signal may contain components corresponding to various heart sound components such as one or more of S1, S2, S3, or S4.

The cardiac sensing circuit 214 can sense a cardiac electrical signal. Examples of the cardiac electrical signal may include surface electrocardiogra (ECG) sensed from electrodes placed on the body surface, subcutaneous ECG sensed from electrodes placed under the skin, intracardiac electrogram (EGM) sensed from the one or more implantable electrodes. The sensing electrodes can be included in or communicatively coupled to the IMD 102 or the WMD 103. The heart rate circuit 216 can detect heart rate of the patient, such as from the signal sensed by the cardiac sensing circuit 214. Alternatively, the heart rate (or pulse rate) can be detected from a cardiac mechanical signal. As to be discussed further below, one or more of the cardiac electrical signal or the heart rate information may be used by the control circuit to construct a representative heart sound segment from which a spectral entropy time series can be derived.

In some examples, the data receiver circuit 210 may receive other physiological or functional signals including, for example, physical activity signal, posture signal, a thoracic or cardiac impedance signal, arterial pressure signal, pulmonary artery pressure signal, left atrial pressure signal, RV pressure signal, LV coronary pressure signal, coronary blood temperature signal, blood oxygen saturation signal, heart sound signal, physiologic response to activity, apnea hypopnea index, one or more respiration signals such as a respiratory rate signal or a tidal volume signal, brain natriuretic peptide (BNP), blood panel, sodium and potassium levels, glucose level and other biomarkers and bio-chemical markers, among others.

The patient symptom receiver 218 may receive information about symptoms of the patient relevant to aortic stenosis. Examples of such symptoms may include: fatigue (e.g., difficulty walking short distances, decline in activity level or duration, or reduced ability to do normal physical activities); shortness of breath or trouble breathing; chest pain; dizziness, fainting or lightheadedness; heart murmur, palpitations or other irregular heartbeats; among others. Some symptoms (e.g., fatigue, shortness or breath, or chest pain) may be prominent or worsen during or after physical activities. Presence and/or degree of patient symptoms may be received as user input such as via the user interface 230. Additionally or alternatively, one or more sensors may sense physiological information correlated to or indicative of the presence and degree of certain symptoms. For example, a thoracic impedance sensor may sense a respiration signal, which can be analyzed to detect respiratory rate and tidal volume that are correlated to or indicative of respiratory symptoms of shortness of breath. In another example, cardiac electrical signals and/or heart rates may be analyzed to detect irregular heartbeats or rhythms related to symptoms of heart murmur or palpitations.

The controller circuit 220 can recognize and track a heart sound component (e.g., S1 or S2 sounds), and detect aortic stenosis using at least the heart sound component. The controller circuit 220 may be implemented as a part of a microprocessor circuit, which may be a dedicated processor such as a digital signal processor, application specific integrated circuit (ASIC), microprocessor, or other type of processor for processing information including physical activity information. Alternatively, the microprocessor circuit may be a general-purpose processor that may receive and execute a set of instructions of performing the functions, methods, or techniques described herein.

The controller circuit 220 may include circuit sets comprising one or more other circuits or sub-circuits, such as a heart sound (HS) recognition circuit 222, an aortic stenosis detector 224, a valve replacement assessment unit 226, and a heart failure detector 228. These circuits may, alone or in combination, perform the functions, methods, or techniques described herein. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

The HS recognition circuit 222 can preprocess the heart sound signal received from the HS sensing circuit 212. In an example, the HS recognition circuit 222 may include a filter or a filter bank to remove or attenuate one or more of low-frequency signal baseline drift, high frequency noise, or other unwanted frequency contents. In an example, the heart sound segment may be band-pass filtered to a frequency range of approximately 5-90 Hz, or approximately 9-90 Hz. In an example, the filter may include a double or higher-order differentiator configured to calculate a double or higher-order differentiation of the heart sound signal.

The HS recognition circuit 222 may recognize a heart sound component from the preprocessed heart sound signal, optionally further using the cardiac electrical signal and the heart rate information received respectively from the cardiac sensing circuit 214 and the heart rate circuit 216. Examples of the heart sound components include S1, S2, S3, and S4 components. S1 and S2 heart sounds generally have a frequency within a range of approximately 10-250 Hz. With a wide interpersonal variability and shifts secondary to technical equipment, S2 generally has a higher frequency than S1. For example, most of S1 power fall within approximately 10-50 Hz and most of S2 fall within approximately 20-70 Hz. Early diastole sound S3 produced by rapid filling of dilated ventricles, and late diastole sound S4 produced by contraction of the left atrium against a non-compliant left ventricle, when present, generally have lower intensity and lower frequencies. The HS recognition circuit 222 may recognize one or more of S1, S2, S3, or S4 heart sounds using a time-domain, amplitude-based approach which includes detecting a maximum signal amplitude or a variation thereof, such as peak heart sound signal power or root-mean-square (RMS) value of the heart sound signal, within a heart sound detection window. The timing of the maximum amplitude may be identified as the timing of the heart sound component. In some examples, the HS recognition circuit 222 may recognize one or more of S1, S2, S3, or S4 heart sounds using a frequency-domain method, such as a power spectrum or a spectral entropy time series of the heart sound signal.

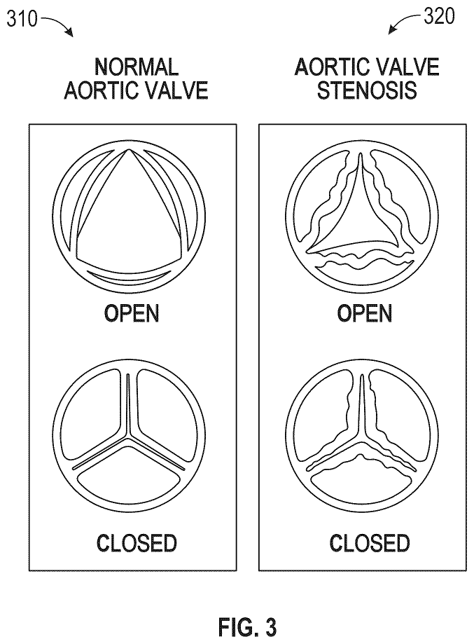

The aortic stenosis detector 224 can detect presence and severity of aortic stenosis based at least in part on the heart sound components recognized by the HS recognition circuit 222. The aortic stenosis detector 224 may include an aortic valve surface area estimator 225 that can estimate a surface area of the aortic value (S_{AV}) or a change in S_{AV} using one or more heart sound components. Aortic stenosis can cause changes in aortic valve anatomy, including reduced valve surface area and thickening and calcification of valve leaflets. Referring now to FIG. 3, the graphs illustrate a normal aortic valve 310 and a stenotic aortic valve 320 in open and closed states. Normal aortic valves in healthy adults have a surface area of approximately 3.0 to 4.0 cm². In patients with symptomatic aortic stenosis, the valve surface area may be less than 0.8 cm². As aortic stenosis develops, minimal pressure gradient is present until the orifice area (the open space defined by valve leaflets) can be less than half of normal. The pressure gradient across a stenotic valve is directly related to the valve orifice area and the transvalvular flow. As the stenotic valve cannot fully open and interfere with the normal blood flow out of the heart, it may lead to heart failure or other cardiac and systemic problems. The restricted blood flow limits oxygen supply to body organs and tissue, causing various symptoms including chest pain, shortness of breath and fainting.

In cardiac catheterization, Gorlin equation (also known as Gorlin formula) has been used to estimate aortic valve surface area (S_{AV}) and to assess the severity of aortic stenosis. One form of Gorlin equation is given as follows:

$${S}_{AV} = Q / ( 44. 3 * \sqrt{Δ P} ) \qquad ( 1 )$$

In Gorlin equation (1) above, AP represents the pressure gradient between aorta and left ventricle (LV), which can be measured at peak systole. AP thus computed is also referred to as peak instantaneous pressure gradient during the period of systolic ejection. Q represents a normalized cardiac output (CO, the volume of blood the heart pumps per minute), which can be computed as a ratio of CO to systolic ejection period (SEP, the accumulated systolic time per minute). As SEP can be determine as a product of per-beat left ventricular ejection time (LVET) and heart rate (HR), the Gorlin equation can be rewritten as follows:

$${S}_{AV} = CO / ( 44. 3 * HR * LVET * \sqrt{Δ P} ) \qquad ( 2 )$$

To estimate S_{AV} or a change in S_{AV} from a baseline aortic stenosis-free state, the aortic valve surface area estimator 225 can estimate LVET using a time interval between S1 and S2 heart sounds (T_{S1-S2}), and estimate aorta-to-LV pressure gradient ΔP using an intensity of S2 heart sound (∥S2∥), such as a peak amplitude or a peak signal power within the S2 detection window. The S1-S2 time interval T_{S1-S2} is directly proportional to the LVET. The intensity of S2 heart sound ∥S2∥ is directly proportional to the peak instantaneous aorta-to-LV pressure gradient ΔP during the period of systolic ejection. During ejection, aortic pressure is slightly lower than LV pressure. In case of aortic stenosis, the stenotic aortic valve adds further resistance to blow flow during systole, which results in a dramatic LV-to-aorta pressure gradient. As the LV begins to relax at the end of ejection, a reversal aorta-to-LV pressure gradient causes the aortic valve slamming shut. As opposed to a normal heart where reversal is happening from a relatively smaller LV-to-aorta difference during systole, in stenotic aortic value, a relatively higher LV-to-aorta pressure gradient during systole causes a relatively larger aorta-to-LV gradient at the time of aortic valve closure. The aortic valve surface area estimator 225 can estimate S_{AV} or a change in S_{AV} from a baseline aortic stenosis-free state using the following HS-based Gorlin formula:

$${S}_{AV} ∝ 1 / HR * {T}_{S 1 - S 2} * \sqrt{‖ S2 ‖} ) \qquad ( 3 )$$

The HS-based Gorlin formula above shows that S_{AV} is in inverse proportion to each of HR, T_{S1-S2}, and a square root of S2 intensity. An increase in S2 intensity and/or a prolonged S1-S2 time interval may lead to a decrease in aortic valve surface area S_{AV}, indicating a presence or elevated severity of aortic stenosis. In an example, to estimate a change in aortic surface area and to detect aortic stenosis, the aortic valve surface area estimator 225 can compute a product metric X=HR*T_{S1-S2}*\sqrt{∥S2∥}, and compare it to a threshold value (X₀) representing said product metric calculated during a baseline aortic stenosis-free state. If the computed product metric X exceeds the threshold X₀ by a specific margin, then the aortic stenosis detector 224 can generate an aortic stenosis indicator indicating a significant decrease in aortic value surface area and that aortic stenosis is present. In some examples, the aortic stenosis detector 224 can further determine a degree or severity of aortic stenosis based on a relative measure (e.g., a difference or a ratio) between the computed product metric X and the baseline product metric value X₀. For example, a higher increase from X₀ corresponds to a higher decrease in aortic valve surface area and more severe aortic stenosis, and vice versa.

The HS-based aortic stenosis indicator may be provided to a user (e.g., a clinician) such as displayed on the user interface 230. In an example, the aortic valve surface area estimator 225 may be commanded to continuously or periodically estimate aortic surface area and track changes thereof, so as to timely capture valve deterioration and thus provide early detection of aortic stenosis. In response to the aortic stenosis indicator indicating a presence of aortic stenosis, an alert can be provided to a patient to follow up with physician for further cardiac performance evaluation (e.g., echocardiogram to assess LV function). The alert can be a visual alert, an audible alert, or a haptic alert (e.g., vibration), among other forms of alert.

In some examples, when the aortic stenosis indicator indicates a presence of aortic stenosis, the controller circuit 220 may trigger the data receiver circuit 210 to receive patient symptoms at the time of the detection of aortic stenosis. As described above, the patient symptoms may be received as subjective input, or objective physiological information sensed by one or more physiological sensors associated with the patient that are correlated to or indicative of functional deterioration associated with or caused by aortic stenosis. Examples of the sensors used for detecting functional deterioration associated with aortic stenosis include a physical activity sensor to detect reduced activity and fatigue, a respiration sensor to detect shortness of breath, a heart rate sensor and/or an electrocardiogram sensor to detect heart murmur or palpitations or other irregular heartbeats, a heart sound sensor to detect an increase in cardiac contractility based on an elevated S1 sound intensity, etc. In some examples, the alert to the patient can be further based on patient symptoms or sensor-indicated patient functional deterioration. The aortic stenosis detector 224 can categorize aortic stenosis severity level based on the HS-based aortic stenosis indicator and the received symptom information.

The HS-based aortic stenosis indicator produced by the aortic stenosis detector 224 may be used to facilitate patient triage and to titrate therapies. The valve replacement assessment unit 226 can generate an indicator of patient candidacy for an aortic valve replacement procedure, such as transcatheter aortic valve replacement (TAVR) or surgical aortic valve replacement (SAVR), based at least in part on the aortic stenosis indicator. The valve replacement candidacy may further be based on patient symptom or physiological information of the patient indicative of functional deterioration associated with the presence of aortic stenosis. For example, if the change in aortic value surface area exceeds a threshold indicating a presence of aortic stenosis, and if the patient symptom or function deterioration satisfies a specific condition (e.g., the change in cardiac contractility which can be estimated based on S1 intensity or a change in S1 intensity exceeds a threshold, or the physiological sensor measurements indicate bouts of increase in Rapid Shallow Breathing Index (RSBI, a ratio of respiratory rate to tidal volume) indicating worsened shortness of breath, irregular heartbeats, or syncope episodes, etc.), then an alert can be provided to a patient or a physician to consider aortic valve correction or replacement procedures.

The heart failure detector 228 can detect a worsening heart failure (WHF) using physiological information (such as collected from a suite of sensors) received from the data receiver circuit. In response to the aortic stenosis indicator indicating a presence of aortic stenosis, the controller circuit 220 can generate a diagnosis of WHF secondary to aortic stenosis, also referred to as “aortic stenosis-triggered” WHF, and provide the diagnosis to the user. Differentiating the “non-aortic stenosis triggered” WHF from “aortic stenosis-triggered” WHF may facilitate triaging heart failure management strategies. For example, for WHF patients with no aortic stenosis or unchanged stenosis severity, routine heart failure treatment (e.g., diuretic up-titration or adjusting electrostimulation) may be administered. If WHF is accompanied by a detected deterioration of aortic stenosis, then an aortic stenosis-triggered WHF is suspected, and interventions such as balloon valvuloplasty or aortic valve replacement may be recommended to reverse valve deterioration.

The user interface 230 may include an input unit and an output unit. In an example, at least a portion of the user interface 230 may be implemented in the external system 105. The input unit may receive user input for programming the data receiver circuit 210 and the controller circuit 220, such as parameters for sensing the heart sound signal, detecting a heart sound component, or detecting aortic stenosis or determining an aortic stenosis severity based on the estimated change in aortic surface area. The input unit may include a keyboard, on-screen keyboard, mouse, trackball, touchpad, touchscreen, or other pointing or navigating devices. The output unit may include a display for displaying the sensed heart sound signal, the representative heart sound segments, the spectral entropy time series, the heart sound metrics, information about the detected physiologic events, and any intermediate measurements or computations, among others. The output unit may also present to a user, such as via a display unit, the therapy titration protocol and recommended therapy, including a change of parameters in the therapy provided by an implanted device, the recommendation to get a device implanted or a procedure (e.g., a valve replacement procedure such as TAVR or SAVR), the initiation or change in a drug therapy, or other treatment options of a patient. The output unit may include a printer for printing hard copies of information that may be displayed on a display unit. The signals and information may be presented in a table, a chart, a diagram, or any other types of textual, tabular, or graphical presentation formats. The presentation of the output information may include audio or other media format. In an example, the output unit may generate alerts, alarms, emergency calls, or other forms of warnings to signal the system user about the detected medical events.

The therapy circuit 240 may be configured to deliver a therapy to the patient, such as in response to the detected presence of aortic stenosis or based on the severity of aortic stenosis. The therapy may be preventive or therapeutic in nature such as to modify, restore, or improve patient cardiac functions. In an example, the therapy circuit 240 may deliver heart failure therapy in response to the heart failure detector 228 detecting WHF. If the detected WHF is determined to be secondary to aortic stenosis (i.e., the “aortic stenosis-triggered” WHF), the therapy circuit 240 may provide treatment options to manage aortic stenosis, including medication to help control the symptoms and lower the chance of developing certain complications, or recommended procedures such as balloon valvuloplasty or aortic valve replacement. If the detected WHF is determined to be “non-aortic stenosis triggered” WHF, then standard heart failure management regimes may be administered, such as drug therapy or electrostimulation. Examples of the therapy may include electrostimulation therapy delivered to the heart, a nerve tissue, other target tissues, a cardioversion therapy, a defibrillation therapy, or drug therapy including delivering drug to the patient. In some examples, the therapy circuit 240 may modify an existing therapy, such as adjust a stimulation parameter (e.g., a stimulation amplitude, frequency, pulse width, electrode configuration, stimulation time) of an electrostimulation therapy, or adjust a drug dosage of a drug therapy.

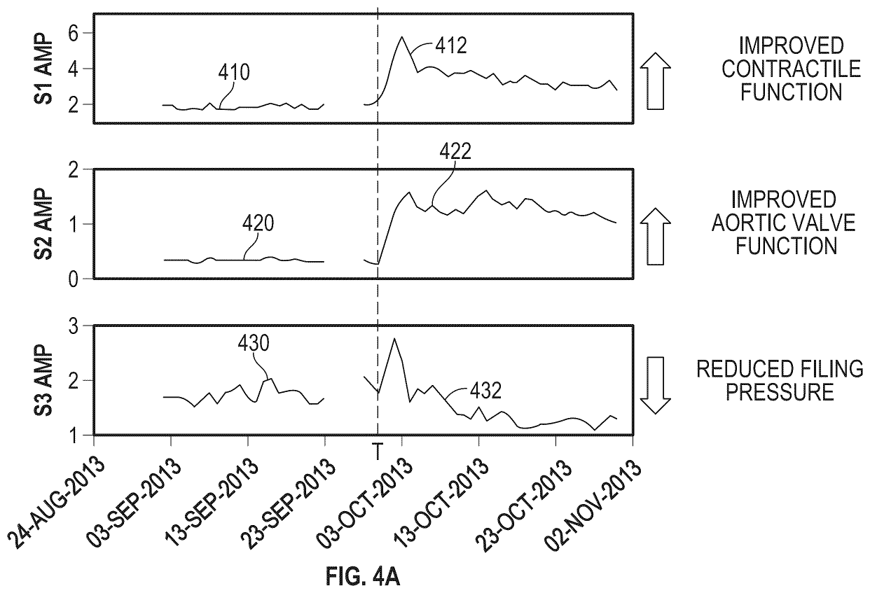

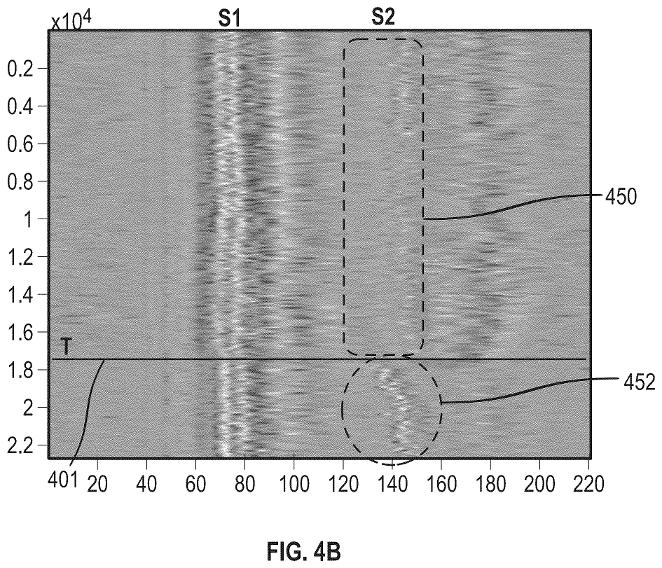

FIGS. 4A and 4B illustrate example changes in heart sounds in an aortic stenosis patient before and after a TAVR procedure. In the illustrated example, various heart sound components, such as generated using the HS recognition circuit 222, are used to evaluate post TAVR improvement in cardiac function. The heart sound components include S1 sound intensity to indicate contractile function of the heart, S2 sound intensity to indicate aortic valve function, and S3 sound intensity to indicate cardiac filling pressure. As shown in FIG. 4A, compared to pre-TAVR (prior to procedure time T) baseline heart sound data, post-TAVR (after the procedure time T) S1 intensity 412 increases substantially from baseline S1 intensity 410, indicating an improved contractile function. Post-TAVR S2 intensity 422 increases substantially from baseline S2 intensity 420, indicating an improved aortic valve function. Post-TAVR S3 intensity 432 also increases substantially from baseline S3 intensity 430, indicating reduced filling pressure and improved diastolic function. FIG. 4B shows a heart sound map to illustrate beat-by-beat changes in heart sound intensity (e.g., amplitude) before and after TAVR procedure at time T, shown as a horizontal time line 401. A heart sound map represents a plurality of heart sound segments stacked together along the y-axis, indexed by the number of heart sound segments (thus correspond to time). The plurality of heart sound segments are each extracted from multiple cardiac cycles, and are aligned with respect to a fiducial point (the origin of the x-axis), such as the R wave of respective cardiac cycle. Each heart sound segment (on the x-axis) has the signal amplitudes color-coded or gray scale-coded to show the changes in amplitude over time. As illustrated in FIG. 4B, post-TAVR S2 amplitude 452 substantially increases from pre-TAVR baseline S2 amplitude 450, indicating an improved aortic valve function following the TAVR procedure.

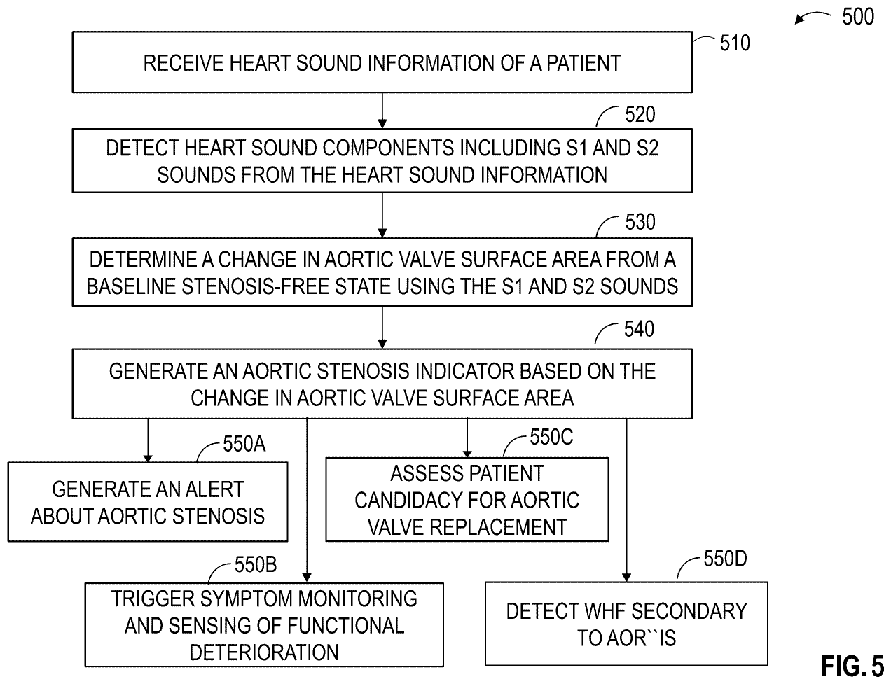

FIG. 5 is a flowchart illustrating an example method 500 of managing a valvular heart disease such as aortic stenosis. The method 500 may be implemented and executed in an ambulatory medical device such as an implantable or wearable medical device, or in a remote patient management system. In an example, the method 500 may be implemented in and executed by the IMD 102 or the WMD 103, the external system 105, or the aortic stenosis detection system 200.

The method 500 begins at 510 to receive heart sound information sensed from a patient. The heart sound information may be sensed using a sensor associated with or included in an ambulatory or wearable device. In some examples, endocardial acceleration signals sensed from inside the heart may be used to analyze heart sounds. Other physiologic information may also be received, including a cardiac electrical signal such as an electrocardiogramar an intracardiac electrogram (EGM), heart rate, signals indicative of cardiac mechanical activity including, for example, thoracic or cardiac impedance signal, arterial pressure signal, pulmonary artery pressure signal, left atrial pressure signal, RV pressure signal, LV pressure signal, heart sounds or endocardial acceleration signal, physiologic response to activity, apnea hypopnea index, one or more respiration signals such as a respiration rate signal or a tidal volume signal, among others.

At 520, heart components including S1 sound and S2 sound can be detected from the received heart sound information. Time-domain methods or frequency-domain methods may be used to detect S1 and S2 sounds, as described above with respect to FIG. 2.

At 530, a change in aortic valve surface area from a baseline stenosis-free state can be determined using features of the detected S1 and S2 sounds. In one example, the aortic valve surface area (S_{AV}) may be estimated using a modified Gorlin equation, where the left ventricular ejection time (LVET) can be approximated by a time interval between S1 and S2 heart sounds (T_{S1-S2}), and the aorta-to-LV pressure gradient ΔP can be approximated by S2 sound intensity (∥S2∥), such as peak amplitude or peak signal power within the S2 detection window, as described above with respect to FIG. 2. The aortic valve surface areas S_{AV} can be determined to be in inverse proportion to HR, T_{S1-S2}, and ∥S2∥.

At 540, an aortic stenosis indicator may be generated based on the change in aortic valve surface area. An increase in S2 intensity and/or a prolonged S1-S2 time interval may cause a decrease in aortic valve surface area S_{AV}, indicating a presence or elevated severity of aortic stenosis. In an example, the product metric X=HR*T_{S1-S2}*\sqrt{∥S2∥} can be compared to a threshold value (X₀) representing said product determined during a baseline aortic stenosis-free state, and the comparison result can be used to estimate a change in aortic surface area. If the product metric X exceeds the threshold X₀ by a specific margin, then a significant decrease in aortic value surface area is detected, indicating aortic stenosis is present.

The aortic stenosis indicator generated at 540 can be provided to a user (e.g., the patient or a clinician managing the patient) or a process executable by the medical-device system. In one example, the HS-based aortic surface area can be continuously or periodically monitored to timely capture significant changes in aortic surface area and therefore produce an early detection of aortic stenosis. At 550A, in response to the aortic stenosis indicator indicating a presence of aortic stenosis, an alert can be provided to a patient to follow up with physician for further cardiac performance evaluation (e.g., echocardiogram to assess LV function). Additionally or alternatively, at 550B, the detection of a presence of aortic stenosis may trigger patient symptom monitoring, or sensing of functional deterioration in the patient associated with or caused by aortic stenosis such as via one or more sensors (e.g., a physical activity sensor to detect reduced activity and fatigue, a respiration sensor to detect shortness of breath, a heart rate sensor and/or an electrocardiogram sensor to detect heart murmur or palpitations or other irregular heartbeats, a heart sound sensor to detect an increase in cardiac contractility based on an elevated S1 sound intensity, etc.). The alert to the patient can be further based on patient symptoms or measured functional deterioration.

The aortic stenosis indicator may alternatively or additionally be used in medical diagnosis and/or for titrating therapies. At 550 C, patient candidacy for aortic valve replacement procedure (e.g., TAVR or SAVR) may assessed based at least in part on the aortic stenosis indicator. The valve replacement candidacy may further be based on patient symptom or physiological information of the patient indicative of functional deterioration associated with the presence of aortic stenosis. For example, in the presence of aortic stenosis, if patient symptom or function deterioration satisfies a specific condition (e.g., the change in cardiac contractility which can be estimated based on S1 intensity exceeds a threshold, or the physiological sensor measurements indicate bouts of increase in RSBI indicating worsened shortness of breath, irregular heartbeats, or syncope episodes, etc.), then an alert can be provided to a patient or a physician to consider TAVR or SAVR procedure. Additionally or alternatively, at 550D, worsening heart failure (WHF) may be detected using patient physiological information sensed from multiple sensors. If the WHF is accompanied by a presence of aortic stenosis as indicated by decrease HS-based aortic valve surface area, a diagnosis of WHF secondary to aortic stenosis (i.e., the “aortic stenosis-triggered” WHF) may be made, and recommendation can be made to the user to titrate therapy. For example, if WHF is detected by aortic stenosis is not present or its severity is unchanged, then routine heart failure management strategies (e.g., diuretic up-titration or adjusting electrostimulation) may be administered. If WHF is accompanied by aortic stenosis or an elevated severity, then “aortic stenosis-triggered” WHF is suspected, and the interventions such as aortic valve replacement or balloon valvuloplasty may be considered to reverse valve deterioration.

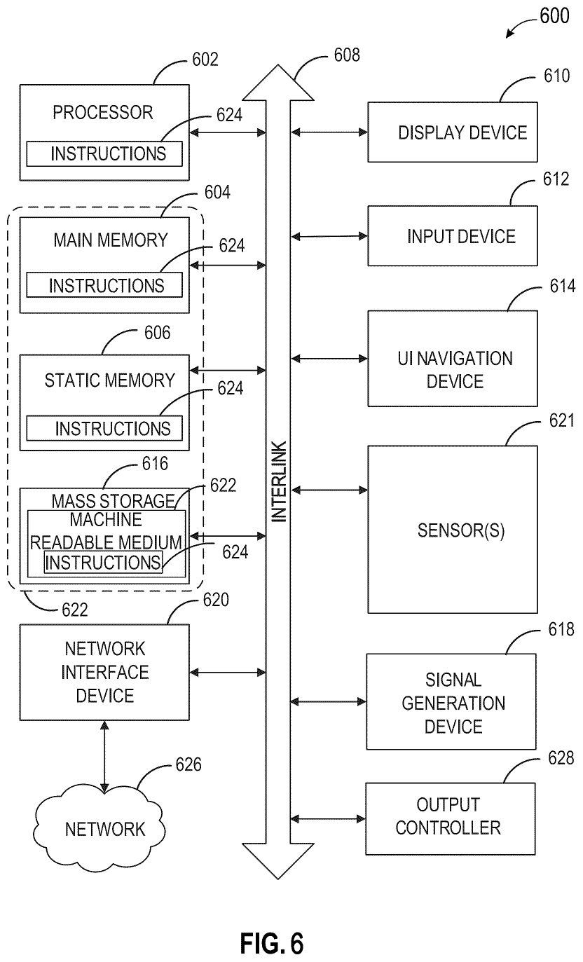

FIG. 6 illustrates generally a block diagram of an example machine 600 upon which any one or more of the techniques (e.g., methodologies) discussed herein may perform. Portions of this description may apply to the computing framework of various portions of the IMD 102, the WMD 103, the external system 105, or the aortic stenosis detection system 200.

In alternative embodiments, the machine 600 may operate as a standalone device or may be connected (e.g., networked) to other machines. In a networked deployment, the machine 600 may operate in the capacity of a server machine, a client machine, or both in server-client network environments. In an example, the machine 600 may act as a peer machine in peer-to-peer (P2P) (or other distributed) network environment. The machine 600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 600 may include a hardware processor 602 (e.g., a central processing unit (CPU), a graphics processing unit (GPU), a hardware processor core, or any combination thereof), a main memory 604 and a static memory 606, some or all of which may communicate with each other via an interlink (e.g., bus) 608. The machine 600 may further include a display unit 610 (e.g., a raster display, vector display, holographic display, etc.), an alphanumeric input device 612 (e.g., a keyboard), and a user interface (UI) navigation device 614 (e.g., a mouse). In an example, the display unit 610, input device 612 and UI navigation device 614 may be a touch screen display. The machine 600 may additionally include a storage device (e.g., drive unit) 616, a signal generation device 618 (e.g., a speaker), a network interface device 620, and one or more sensors 621, such as a global positioning system (GPS) sensor, compass, accelerometer, or other sensor. The machine 600 may include an output controller 628, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 616 may include a machine readable medium 622 on which is stored one or more sets of data structures or instructions 624 (e.g., software) embodying or utilized by any one or more of the techniques or functions described herein. The instructions 624 may also reside, completely or at least partially, within the main memory 604, within static memory 606, or within the hardware processor 602 during execution thereof by the machine 600. In an example, one or any combination of the hardware processor 602, the main memory 604, the static memory 606, or the storage device 616 may constitute machine readable media.

While the machine-readable medium 622 is illustrated as a single medium, the term “machine readable medium” may include a single medium or multiple media (e.g., a centralized or distributed database, and/or associated caches and servers) configured to store the one or more instructions 624.

The term “machine readable medium” may include any medium that is capable of storing, encoding, or carrying instructions for execution by the machine 600 and that cause the machine 600 to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine readable medium with a plurality of particles having invariant (e.g., rest) mass. Accordingly, massed machine-readable media are not transitory propagating signals. Specific examples of massed machine-readable media may include: non-volatile memory, such as semiconductor memory devices (e.g., Electrically Programmable Read-Only Memory (EPROM), Electrically Erasable Programmable Read-Only Memory (EPSOM)) and flash memory devices; magnetic disks, such as internal hard disks and removable disks; magneto-optical disks; and CD-ROM and DVD-ROM disks.

The instructions 624 may further be transmitted or received over a communication network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as WiFi®, IEEE 802.16 family of standards known as WiMax®), IEEE 802.15.4 family of standards, peer-to-peer (P2P) networks, among others. In an example, the network interface device 620 may include one or more physical jacks (e.g., Ethernet, coaxial, or phone jacks) or one or more antennas to connect to the communication network 626. In an example, the network interface device 620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 600, and includes digital or analog communications signals or other intangible medium to facilitate communication of such software.

Various embodiments are illustrated in the figures above. One or more features from one or more of these embodiments may be combined to form other embodiments.

The method examples described herein can be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should therefore be determined with references to the appended claims, along with the full scope of equivalents to which such claims are entitled.

What is claimed is:

1. A medical-device system for managing a valvular heart disease, comprising:
a data receiver circuit configured to receive heart sound information sensed from a patient; and
a controller circuit, comprising a stenosis detector circuit configured to:
detect heart sound components including S1 sound and S2 sound from the received heart sound information;
determine a change in aortic valve surface area from a baseline stenosis-free state using the detected S1 and S2 sounds; and
generate an aortic stenosis indicator based at least in part on the determined change in aortic valve surface area,
wherein the controller circuit is configured to provide the aortic stenosis indicator to a user or a process executable by the medical-device system.

2. The medical-device system of claim 1, wherein to determine the change in aortic valve surface area includes to detect a decrease in aortic valve surface area from the baseline stenosis-free state,
wherein the stenosis detector is configured to generate the aortic stenosis indicator indicating a presence of aortic stenosis in response to the decrease in aortic value surface area exceeding a threshold.

3. The medical-device system of claim 1, wherein to determine the change in aortic valve surface area, the stenosis detector is configured to:
generate an estimate of a left ventricular ejection time using a time interval between the detected S1 and S2 sounds, and an estimate of an aorta-to-left ventricular pressure gradient using an intensity of the S2 sound; and
determine the aortic valve surface area based on the estimate of the aorta-to-left ventricular pressure gradient and the estimate of the left ventricular ejection time.

4. The medical-device system of claim 3, wherein the stenosis detector is configured to determine the aortic valve surface area to be inversely proportional to the estimate of the left ventricular ejection time, and inversely proportional to a square root of the estimate of the aorta-to-left ventricular pressure gradient.

5. The medical-device system of claim 1, wherein the controller circuit is configured to generate an alert to the user in response to the aortic stenosis indicator indicating a presence of aortic stenosis.

6. The medical-device system of claim 1, wherein the controller circuit is configured to, in response to the aortic stenosis indicator indicating a presence of aortic stenosis:
trigger the data receiver circuit to receive symptom or physiological information of the patient indicative of functional deterioration associated with the presence of aortic stenosis; and
categorize a stenosis severity level based on the aortic stenosis indicator and the received symptom or physiological information.

7. The medical-device system of claim 6, comprising a user interface coupled to data receiver circuit, the user interface configured to receive a user input of symptom in response to the indication of the presence of aortic stenosis.

8. The medical-device system of claim 6, wherein the triggered received physiological information includes an intensity of S1 sound indicative of cardiac contractility.

9. The medical-device system of claim 6, comprising one or more sensors coupled to data receiver circuit and configured to sense physiological information in response to the indication of the presence of aortic stenosis, the one or more sensors including at least one of a physical activity sensor, a respiration sensor, a heart rate sensor, or an electrocardiogram sensor.

10. The medical-device system of claim 1, wherein the controller circuit is configured to generate an indicator of patient candidacy for aortic valve replacement procedure based at least in part on the aortic stenosis indicator.

11. The medical-device system of claim 10, wherein the controller circuit is configured receive symptom or physiological information of the patient indicative of functional deterioration associated with the aortic stenosis, and to generate the indicator of patient candidacy for aortic valve replacement further based on the received symptom or physiological information.

12. The medical-device system of claim 1, wherein the controller circuit comprises a heart failure detector circuit configured to detect worsening heart failure (WHF) using physiological information received from the data receiver circuit, and in response to the aortic stenosis indicator indicating a presence of aortic stenosis, the controller circuit is configured to:
generate a diagnosis of WHF secondary to aortic stenosis; and
generate a recommendation to titrate therapy based on the diagnosis of WHF secondary to aortic stenosis.

13. A method of managing a valvular heart disease using a medical-device system, the method comprising:
receiving heart sound information sensed from a patient; and
detecting heart sound components including S1 sound and S2 sound from the received heart sound information;
determining, via a stenosis detector circuit, a change in aortic valve surface area from a baseline stenosis-free state using the detected S1 and S2 sounds;
generating an aortic stenosis indicator based at least in part on the determined change in aortic valve surface area; and
providing the aortic stenosis indicator to a user or a process executable by the medical-device system.

14. The method of claim 13, wherein determining the change in aortic valve surface area includes detecting a decrease in aortic valve surface area from the baseline stenosis-free state,
wherein the aortic stenosis indicator indicates a presence of aortic stenosis when the decrease in aortic value surface area exceeds a threshold.

15. The method of claim 13, wherein determining the change in aortic valve surface area includes:
generating an estimate of a left ventricular ejection time using a time interval between the detected S1 and S2 sounds, and generating an estimate of an aorta-to-left ventricular pressure gradient using an intensity of the S2 sound; and
determining an aortic valve surface area based on the estimate of the aorta-to-left ventricular pressure gradient and the estimate of the left ventricular ejection time.

16. The method of claim 13, comprising generating an alert to the user in response to the aortic stenosis indicator indicating a presence of aortic stenosis.

17. The method of claim 13, comprising, in response to the aortic stenosis indicator indicating a presence of aortic stenosis:
receiving symptom or physiological information of the patient indicative of functional deterioration associated with the presence of aortic stenosis; and
categorizing a stenosis severity level based on the aortic stenosis indicator and the received symptom or physiological information.

18. The method of claim 17, wherein receiving the symptom or physiological information of the patient includes receiving a user input of symptom, or sensing physiological information using one or more sensors including at least one of a heart sound sensor, physical activity sensor, a respiration sensor, a heart rate sensor, or an electrocardiogram sensor.

19. The method of claim 13, comprising generating, and providing to a user, an indicator of patient candidacy for aortic valve replacement procedure based at least in part on the aortic stenosis indicator.

20. The method of claim 13, comprising:
detecting worsening heart failure (WHF) using physiological information; and
in response to the aortic stenosis indicator indicating a presence of aortic stenosis, generating a diagnosis of WHF secondary to aortic stenosis, and generating a recommendation to titrate therapy based on the diagnosis of WHF secondary to aortic stenosis.
